SEQUENCE LISTING (10 sequences)

SEQ ID NO: 1 - DNA, 33 bp, Artificial Sequence
  Description of Artificial Sequence Synthetic primer
gccggccagtactggccactccgcatcgtgcag

SEQ ID NO: 2 - DNA, 33 bp, Artificial Sequence
  Description of Artificial Sequence Synthetic primer
cggccggtcatgaccggtgaggcgtagcacgtc

SEQ ID NO: 3 - DNA, 99 bp, Homo sapiens
gccacctgctattgccgaaccggccgttgtgctacccgtgagtccctctccggggtgtgtgaaatcagtggccgcctctacagactctgctgtcgctga

SEQ ID NO: 4 - DNA, 99 bp, Homo sapiens
  CDS (1)..(96)
gccacctgctattgtaggacaggccggtgtgctacgagagagagcctctcaggggtgtgcgaaatttccggtcgcctctaccgtttgtgttgccgctga

SEQ ID NO: 5 - protein, 32 aa, Homo sapiens
ATCYCRTGRCATRESLSGVCEISGRLYRLCCR

SEQ ID NO: 6 - DNA, 99 bp, Homo sapiens
  CDS (1)..(96)
gccacctgctactgcaggaccggccgctgcgccaccagggagagcctctccggggtgtgcgaaatctccggccgcctctacaggctctgctgccgctga

SEQ ID NO: 7 - DNA, 4345 bp, Artificial Sequence
  Description of Artificial Sequence Synthetic construct
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcatgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcacttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcataagagtcacttcacgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagcccacgtgagcgagtcggagatgaggttcagggacaggcagtgccagcgggaggtgcaggacagcccgctggacgcgtgccggcaggtgctcgaccggcagctcaccggccgggagaggttccagccgatgttccgccgcccgggcgcgctcggcctgcggatgcagtgctgccagcagctgcaggacgtgagccgcgagtgccgctgcgccgccatccgccggatggtgaggagctacgaggagagcatgccgatgcccctggagcaaggctggtcgtcgtcgtcgtcggagtactacggcggcgaggggtcgtcgtcggagcaggggtactacggcgaggggtcgtcggaggagggctactacggcgagcagcagcagcagccggggatgacccgcgtgaggctgaccagggcgaggcagtacgcggcgcagctgccgtcgatgtgccgggttgagccccagcagtgcagcatcttcgccgccggccagtacgacgacgacgacaaggccacctgctattgtaggacaggccggtgtgctacgagagagagcctctcaggggtgtgcgaaatttccggtcgcctctaccgtttgtgttgccgctgaactcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcgccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgatccatggcgatgcctgcttgccgaatatcatggtggaaaatggccgcttttctggattcaacgactgtggctggctgggtgtggcggaccgctatcaggacatagcgttggatacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 8 - DNA, 678 bp, Artificial Sequence
  CDS (1)..(675)
  Description of Artificial Sequence Synthetic construct
atggcatccataaatcgccccatagttttcttcacagtttgcttgttcctcttgtgcgatggctccctagcccacgtgagcgagtcggagatgaggttcagggacaggcagtgccagcgggaggtgcaggacagcccgctggacgcgtgccggcaggtgctcgaccggcagctcaccggccgggagaggttccagccgatgttccgccgcccgggcgcgctcggcctgcggatgcagtgctgccagcagctgcaggacgtgagccgcgagtgccgctgcgccgccatccgccggatggtgaggagctacgaggagagcatgccgatgcccctggagcaaggctggtcgtcgtcgtcgtcggagtactacggcggcgaggggtcgtcgtcggagcaggggtactacggcgaggggtcgtcggaggagggctactacggcgagcagcagcagcagccggggatgacccgcgtgaggctgaccagggcgaggcagtacgcggcgcagctgccgtcgatgtgccgggttgagccccagcagtgcagcatcttcgccgccggccagtacgacgacgacgacaaggccacctgctattgtaggacaggccggtgtgctacgagagagagcctctcaggggtgtgcgaaatttccggtcgcctctaccgtttgtgttgccgctga

SEQ ID NO: 9 - protein, 225 aa, Artificial Sequence
  Description of Artificial Sequence Synthetic construct
MASINRPIVFFTVCLFLLCDGSLAHVSESEMRFRDRQCQREVQDSPLDACRQVLDRQLTGRERFQPMFRRPGALGLRMQCCQQLQDVSRECRCAAIRRMVRSYEESMPMPLEQGWSSSSSEYYGGEGSSSEQGYYGEGSSEEGYYGEQQQQPGMTRVRLTRARQYAAQLPSMCRVEPQQCSIFAAGQYDDDDKATCYCRTGRCATRESLSGVCEISGRLYRLCCR

SEQ ID NO: 10 - protein, 32 aa, Homo sapiens; identical to SEQ ID NO: 5

(19) United States
(12) Patent Application Publication
(10) Pub. No.: US 2009/0156486 A1
(43) Pub. Date: Jun. 18, 2009

(54) PRODUCTION OF HUMAN DEFENSINS IN PLANT CELLS

(76) Inventors: Ning Huang, Davis, CA (US); Deshui Zhang, Sacramento, CA (US)

Correspondence Address: ARENT FOX LLP, 1050 CONNECTICUT AVENUE, N.W., SUITE 400, WASHINGTON, DC, 20036, US

(21) Appl. No.: 12/096,860

(22) PCT Filed: Dec. 14, 2006
(86) PCT No.: PCT/US06/47606

§ 371 Date: Nov. 3, 2008

Related U.S. Application Data
(60) Provisional application No. 60/750,375, filed on Dec. 15, 2005.

(51) Int. Cl. A61K 38/17; A01H 5/00; C12N 15/82; C07H 21/04; C12N 15/63

(52) U.S. Cl. 514/12; 800/298; 800/320.2; 800/320.1; 800/320; 800/320.3; 800/288; 536/23.6; 435/320.1

(57) ABSTRACT

The present invention relates, generally, to the production of human defensins from plant cells. Heterologous DNA comprising genes encoding one or more desired human defensins are introduced into plant cells. The one or more human defensins can be recombinantly-produced in the plant cells, purified from the plant cells, and used as an anti-infective agent, a wound treatment agent, a skin care agent, or an agent in the treatment of various intestinal diseases, including irritable bowel disease (IBD), ulcerative colitis, and Crohn's disease. The recombinantly-produced α-defensins can be provided as oral and topical formulations.

This application claims priority from U.S. Provisional Application No. 60/750,375, which was filed on Dec. 15, 2005, the contents of which are incorporated herein by reference.

### BACKGROUND OF THE INVENTION

1. Field of the Invention

The present invention relates to the production of human defensins, and preferably to the recombinant production of human a-defensins in plant cells. The present invention also relates to recombinantly-produced human defensins, compositions containing them, and their use in the treatment of various conditions, especially as topical agents for skin care and wound treatment, and in oral formulations for treatment of conditions including intestinal diseases.

2. Description of the Related Art

Virtually all species of vertebrates and invertebrates produce peptides that have broad-spectrum antibiotic activity. A major class of antimicrobial peptides is defensins. Currently, three types of defensins are known—α, β, and θ. Humans, however, appear to produce only α- and β-defensins. To date, six α-defensins and four β-defensins have been characterized, with 31 human defensin genes identified in humans. While β-defensins are expressed at multiple sites in the gastrointestinal tract, α-defensins are largely expressed in the small intestine.

Mature human defensins contain between 30 and 40 amino acid residues and each defensin carries 3 disulfide bonds. Human defensins are effective broad spectrum antimicrobials, particularly in high concentration and low ionic strength. Among the six human a-defensins, four are expressed in neutrophils and two are primarily expressed in Paneth cells. Paneth cells are secretory epithelial cells and the major source of antimicrobial peptides in the small intestine, including human α-defensins 5 and 6 (HD5 and HD6). Among the proteins secreted by Paneth cells, the most abundant ones are HD5 and HD6. Between the two, HD5 is more abundant and much better studied. Paneth cells, located at the base of the crypts of the small intestine, secrete antimicrobial peptides and proteins that contribute to the innate immune defense of the small intestine. Paneth cell products limit the number of commensal bacteria and impact on their composition.

Paneth cells also express the pattern recognition receptor NOD2 and mutations in the Nod2 gene are associated with the clinical phenotype of small intestinal Crohn's disease. All patients with ileal Crohn's disease, but particularly those with Nod2 mutations, are characterized by a diminished expression of both α-defensins, HD5 and HD6 (Wehkamp et al., Gut 53: 1658-64 (2004) and Wehkamp et al PNAS 102:18129-18134 (2005)).

Administration of a-defensins is a potential treatment of intestinal diseases such as Crohn's disease. Thus, there is a need to economically produce human a-defensins that can be used in the treatment of such intestinal diseases.

Plant cells have been studied for the manufacture of human β-defensins. Cai et al. transformed wheat plants with a vector encoding the human β-defensin 2 (HBD-2) gene. J. Sichuan Univ. 34: 385-89 (2003). However, there has been no documented use of plant cells to produce other β-defensins, or human α-defensins.

### SUMMARY OF THE INVENTION

The present invention relates to the production of human defensins, and preferably to the recombinant production of human α-defensins in plant cells. The present invention also relates to recombinantly-produced human defensins, and their use in the treatment of various conditions. There is an unmet need in the art for such compositions and methods.

The present invention meets the unmet needs in the art by providing methods for producing human defensins in plant cells, plant seeds containing human defensins, and compositions comprising recombinant human defensins.

One aspect of the invention comprises a method of transforming a plant cell, preferably a monocot plant such as rice, by incorporating a polynucleotide segment that encodes one or more human defensins.

Another aspect of the invention comprises administering to a subject with an intestinal disease, a composition comprising one or more human defensins produced from plant cells.

A further aspect of the invention comprises administering one or more human defensins produced from plant cells as a topical formulation to a subject as an anti-infective, e.g. for the purpose of wound care.

Another aspect of the invention comprises administering one or more human defensins produced from plant cells as a topical formulation to a subject topically for skin care.

An additional aspect of the invention comprises a method of producing a defensin in plant seeds, comprising the steps of (a) transforming a plant cell with a chimeric gene comprising (i) a maturation-specific protein promoter from a plant, (ii) a first nucleic acid sequence, operably linked to the promoter, encoding a seed-specific signal sequence capable of targeting a polypeptide linked thereto to seed endosperm, and (iii) a second nucleic acid sequence, linked in translation frame with the first nucleic acid sequence, encoding a defensin, wherein the first nucleic acid sequence and the second nucleic acid sequence together encode a fusion protein comprising an N-terminal signal sequence and the defensin; (b) growing a plant from the transformed plant cell for a time sufficient to produce seeds containing the defensin; and (c) harvesting the seeds from the plant.

Another aspect of the invention comprises a method of producing at least one defensin, the method comprising a) providing a plant cell transformed with a vector containing a promoter and a gene, operably linked to the promoter, encoding a defensin, b) growing a plant from the transformed plant cell for a time sufficient to produce seeds, c) harvesting the mature seeds, and optionally d) purifying the desired defensin from the seeds or seed product.

Another aspect of the invention relates to a chimeric gene comprising (i) a promoter that is active in plant cells; (ii) a first nucleic acid sequence, operably linked to the promoter, encoding a seed storage protein; and (iii) a second nucleic acid sequence, operably linked to the promoter, encoding a defensin; wherein the first and second nucleic acid sequences are linked in translation frame and together encode a fusion protein comprising the storage protein and the defensin.

A still further aspect of the invention relates to a vector comprising (i) a promoter from a monocot plant gene that has upregulated activity during seed maturation, (ii) a first DNA sequence, operably linked to said promoter, encoding a monocot plant seed-specific signal sequence capable of targeting a polypeptide linked thereto to monocot plant seed endosperm, and (iii) a second DNA sequence, linked in translation frame with the first DNA sequence, encoding a defensin, wherein the first DNA sequence and the second DNA sequence together encode a fusion protein comprising an N-terminal signal sequence and the defensin.

Yet another aspect of the invention comprises a method of producing seeds that express a defensin and a seed storage protein as a fusion partner, the method comprising (a) transforming a plant cell with a chimeric gene comprising: (i) a promoter that is active in plant cells; (ii) a first nucleic acid sequence, operably linked to the promoter, encoding a seed storage protein; (iii) a second nucleic acid sequence, operably linked to the promoter, encoding a defensin; and (iv) optionally a signal sequence, preferably a seed-specific signal sequence, wherein the first and second nucleic acid sequences and the optional signal sequence are linked in translation frame and together encode a fusion protein comprising the storage protein, the defensin and the optional signal sequence; (b) growing a plant from the transformed plant cell for a time sufficient to produce seeds containing the fusion protein; and (c) harvesting the seeds from the plant.

Other novel features and advantages of the present invention will become apparent to those skilled in the art upon examination of the following or upon learning by practice of the invention.

### BRIEF DESCRIPTION OF THE DRAWINGS

FIG. 1A shows the nucleotide sequence alignment between the native human defensin 5 (HD5) gene and the codon optimized HD5 gene HD5V1. FIG. 1B shows the nucleotide sequence alignment between the native human defensin 5 (HD5) gene and the codon optimized HD5 gene HD5V2. Native HD5=the native human HD5 gene sequence; Codon-optHD5V1 and codon-optHD5V2=two versions of codon-optimized HD5 gene sequences; Amino acids=the translated amino acid residues from their above triplet genetic codons. The vertical lines show the identity of nucleotides between the HD5 gene sequences before and after codon optimization. The numbers represent the position of the nucleotides in the gene sequence. The triplet codons aligned by the shading rectangles show the degenerate codons used in the native and codon-optimized human defensin gene sequences.

FIG. 2 shows schematic diagrams of the gene constructs used in rice transformation for expression of the human defensin 5 (HD5) gene. pVB1, 2, 3, 6, and 12 represent five different plasmid constructs. Gt1 promoter=rice glutelin gene promoter; SIP rice glutelin protein signal peptide; Globulin=rice globulin gene; Lysozyme=codon-optimized human lysozyme gene for mature protein; HD5V1 and HD5V2=codon-optimized HD5 gene sequences; T-Nos=nopaline synthase terminator; EK=enterokinase protease cleavage site; W=amino acid residue tryptophan for N chloro-succinimide cleavage site.

FIGS. 3A-3E show the DNA nucleotide sequence of plasmid pVB1

FIGS. 4A-4B show the DNA nucleotide sequence of fused genes with translated amino acid residues of pVB1.

FIG. 5 shows the translated amino acid sequences of fused proteins including the Gt1 signal peptide, globulin protein, enterokinase cleavage site, and human defensin 5 peptide of pVB1.

FIG. 6 shows the translated amino acid sequence of the human defensin 5 peptide.

FIG. 7 shows the results of an SDS-PAGE analysis of protein extracts from rice grains. M=protein molecular weight marker in kD; Bengal=non-transgenic rice cultivar; VB1-409, 412, 413, and 502=transgenic lines produced by transforming Bengal with plasmid pVB1. Ten pooled seeds from each line were extracted with 5 ml of total protein extraction buffer (65 mM Tris, pH6.8, 2% SDS, 2% BME). 10 μl of protein extract from each line was loaded. Electrophoresis was carried out on a 4-20% Tris-Glycine SDS-PAGE gels (Invitrogen), and the gel was stained with the LabSafe GEL Blue (G-Biosciences, St. Louis, Mo.). The arrowhead indicates the recombinant protein band.

FIG. 8 shows the quantification of recombinant defensin fusion proteins expressed in rice grains. M=protein molecular weight marker in kD; Bengal=non-transgenic rice cultivar; rLys=purified recombinant human lysozyme from rice grains; VB1-392-18, VB1-408-24, VB1-412-18, and VB1-413-9=four R1 seeds from different transgenic lines. Each seed was cut in half transversely, and the embryo-less half-seeds were extracted with 500 μl of protein extraction buffer. 10 μl of protein extract from each half-seed was loaded on a 4-20% Tris-Glycine SDS-PAGE gel (Invitrogen), and the gel was stained with a Coomassie Blue solution. The arrowhead indicates the protein band corresponding to the Globulin-defensin fusion protein. Estimates of the expression level of the rHD5-globulin fusion protein was made based on the intensity of the target bands using recombinant human lysozyme as a standard, using the Kodak 1D image analysis program.

FIGS. 9A and 9B show Western blot analyses for human defensin (FIG. 9A) and rice globulin (FIG. 9B) in rice transgenic plants. M=protein molecular weight marker in kD; Bengal=non-transgenic rice cultivar; all of the other lanes represent protein extracts from different R1 seeds expressing recombinant defensin fusion protein. Each seed was cut in half transversely, and the embryo-less half-seeds were extracted with 500 μl of protein extraction buffer. Protein extract was resolved on a 4-20% Tris glycine SDS-PAGE gel (Invitrogen) and transferred to nitrocellulose membrane using a Mini Trans-Blot Cell (Bio-Rad). The membrane was probed with anti-human defensin 5-α (FIG. 9A), and anti-rice globulin (FIG. 9B), respectively, followed by sequent incubation with secondary antibody (anti-rabbit HRP, Rockland) and ECL reagent (Pierce). The membrane was exposed to CL-Xposure (Pierce) autoradiography film for detection. The arrow indicates the protein bands corresponding to Globulin-defensin fusion proteins.

FIG. 10 shows the results of an SDS-PAGE analysis of individual R1 seeds. M=protein molecular weight marker in kD; Bengal=non-transgenic rice cultivar; VB1-416-1 to 12=12 randomly selected R1 seeds of transgenic line VB1-416; Pool=equally pooled protein extracts from VB1-416-1 to 12. Each seed was cut in half transversely, and the embryo-less half-seeds were extracted with 500 μl of protein extraction buffer. 10 μl of protein extract from each half seed was loaded on a 16% Tris-Glycine SDS-PAGE gel (Invitrogen), and the gel was stained with the Coomassie Blue solution. The arrowhead indicates the protein band corresponding to the Globulin-defensin fusion protein.

### DETAILED DESCRIPTION OF THE PREFERRED EMBODIMENTS

The present invention relates to methods for the production of defensins, preferably human defensins, and more preferably to methods for producing recombinant production of human α-defensins in plant cells. The present invention also relates to recombinantly-produced defensins, and their use in the treatment of various conditions.

Unless otherwise indicated, all terms used herein have the meanings given below or are generally consistent with same meaning that the terms have to those skilled in the art of the present invention. Practitioners are particularly directed to Sambrook et al., Molecular Cloning: A Laboratory Manual (Second Edition), Cold Spring Harbor Press, Plainview, N.Y. (1989); Ausubel F M et al., Current Protocols in Molecular Biology, John Wiley & Sons, New York, N.Y. (1993); and Gelvin and Schilperoot, eds. Plant Molecular Biology Manual, Kluwer Academic Publishers, The Netherlands (1997), for definitions and terms of the art.

General and specific techniques for producing proteins from plant cells may be obtained from the following applications, each of which is incorporated herein in its entirety by reference: U.S. patent application Ser. No. 09/847,232 (“Plant Transcription Factors and Enhanced Gene Expression”); U.S. patent application Ser. No. 10/077,381 (“Expression of Human Milk Proteins in Transgenic Plants”); U.S. patent application Ser. No. 10/411,395 (“Human Blood Proteins Expressed in Monocot Seeds”); U.S. patent application Ser. No. 10/639,779 (“Production of Human Growth Factors in Monocot Seeds”); U.S. patent application Ser. No. 10/639,781 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”).

The nucleic acids of the invention may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

By “host cell” is meant a cell containing a vector and supporting the replication and/or transcription and/or expression of the heterologous nucleic acid sequence Preferably, according to the invention, the host cell is a plant cell. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed” refers to all seed components, including, for example, the coleoptile and leaves, radicle and coleorhiza, scutulum, starchy endosperm, aleurone layer, pericarp and/or testa, either during seed maturation and seed germination. In the context of the present invention, the term “seed” and “grain” is used interchangeably.

The term “seed product” includes, but is not limited to, seed fractions such as de-hulled whole seed, flour (seed that has been de-hulled by milling and ground into a powder), a seed extract, preferably a protein extract (where the protein fraction of the flour has been separated from the carbohydrate fraction), malt (including malt extract or malt syrup) and/or a purified protein fraction derived from the transgenic grain.

The term “biological activity” refers to any biological activity typically attributed to that protein by those skilled in the art.

“Seed components” refers to carbohydrate, protein, and lipid components extractable from seeds, typically mature seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially up-regulated activity (greater than 25%) during seed maturation.

“Heterologous nucleic acid” refers to nucleic acid which has been introduced into plant cells from another source, or which is from a plant source, including the same plant source, but which is under the control of a promoter that does not normally regulate expression of the heterologous nucleic acid.

“Heterologous peptide or polypeptide” is a peptide or polypeptide encoded by a heterologous nucleic acid. The peptides or polypeptides include defensins, preferably human defensins such as an α-defensin or a β-defensin. α-defensins include, but are not limited to, human α-defensin 1 (HD1), human α-defensin 2 (HD2), human α-defensin 3 (HD3), human α-defensin 4 (HD4), human α-defensin 5 (HD5) and human α-defensin 6 (HD6).

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

The invention provides defensins recombinantly produced in host plant seed. Preferably, the defensin expressed comprises about 1% or greater of the total soluble protein in the seed. Thus, for example, the yield of total soluble protein which comprises the defensin targeted for production can be about 3% or greater, about 5% or greater, about 10% or greater, most preferably about 20% or greater, of the total soluble protein found in the recombinantly engineered plant seed.

Preferably, the defensin constitutes at least 0.01 weight percent of the total protein in the harvested seeds. More preferably, the defensin constitutes at least 0.05 weight percent, most preferably at least 0.1 weight percent, of the total protein in the harvested seeds.

An embodiment of the present invention is a method of producing a defensin in plant seeds, comprising the steps of:

(a) transforming a plant cell with a chimeric gene comprising
(i) a maturation-specific protein promoter from a plant,
(ii) a first nucleic acid sequence, operably linked to the promoter, encoding a seed-specific signal sequence capable of targeting a polypeptide linked thereto to seed endosperm, and
(iii) a second nucleic acid sequence, linked in translation frame with the first nucleic acid sequence, encoding an α-defensin, wherein the first nucleic acid sequence and the second nucleic acid sequence together encode a fusion protein comprising an N-terminal signal sequence and the defensin;

(b) growing a plant from the transformed plant cell for a time sufficient to produce seeds containing the defensin; and

(c) harvesting the seeds from the plant.

Preferably, the plant is a monocot plant. More preferably, the plant is a cereal, preferably selected from the group consisting of rice, barley, wheat, oat, rye, corn, millet, triticale and sorghum.

The promoter is preferably from a maturation-specific monocot plant storage protein or an aleurone- or embryo-specific monocot plant gene. Other promoters may be used, however, and the choice of a suitable promoter is within the skill of those in the art. More preferably, the promoter is a member selected from the group consisting of rice globulins, glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1. Most preferably, the promoter is selected from the group consisting of rice globulin Glb promoter and rice glutelin Gt1 promoter.

The seed-specific signal sequence is preferably from a monocot plant, although other signal sequences that target polypeptides to seed endosperm may be utilized. Preferably, the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and lea. More preferably, the monocot plant seed-specific signal sequence is associated with a gene selected from the group consisting of α-amylase, protease, carboxypeptidase, endoprotease, ribonuclease, DNase/RNase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase. Most preferably, the monocot plant seed-specific signal sequence is a rice glutelin Gt1 signal sequence.

As will be understood by those of skill in the art, in some cases it may be advantageous to use a growth factor-encoding nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., J. CAASS 1: 73-86 (1990)). Changing low G+C content to a high G+C content has been found to increase the expression levels of foreign protein genes in barley grains (Horvath et al., Proc. Natl. Acad. Sci. USA 97: 1914-19, (2000)). If a rice plant is selected, the genes employed in the present invention may be based on the rice gene codon bias (Huang et al., supra) along with the appropriate restriction sites for gene cloning. These codon-optimized genes may be linked to regulatory and secretion sequences for seed-directed expression and these chimeric genes then inserted into the appropriate plant transformation vectors.

Another embodiment of the present invention is a method of producing seeds that express a defensin and a seed storage protein as a fusion partner, the method comprising:

(a) transforming a plant cell with a chimeric gene comprising:
(i) a promoter that is active in plant cells;
(ii) a first nucleic acid sequence, operably linked to the promoter, encoding a seed storage protein;
(iii) a second nucleic acid sequence, operably linked to the promoter, encoding a defensin; and
(iv) optionally a signal sequence, preferably a seed-specific signal sequence,
   wherein the first and second nucleic acid sequences and the optional signal sequence are linked in translation frame and together encode a fusion protein comprising the storage protein, the defensin and the optional signal sequence;

(b) growing a plant from the transformed plant cell for a time sufficient to produce seeds containing the fusion protein; and

(c) harvesting the seeds from the plant.

The promoter and signal sequence may be selected from those discussed supra. The type of promoter and signal sequence is not critical to this embodiment of the invention. Preferably, the signal sequence targets the attached fusion protein to a location such as an endosperm cell, more preferably an endosperm-cell subcellular compartment or tissue, such as an intracellular vacuole or other protein storage body, chloroplast, mitochondria, or endoplasmic reticulum, or extracellular space, following secretion from the host cell.

The seed storage protein is preferably from a monocot plant. Preferably, the seed storage protein is selected from the group consisting of rice globulins, rice glutelins, oryzins, prolamines, barley hordeins, wheat gliadins and glutenins, maize zeins and glutelins, oat glutelins, sorghum kafirins, millet pennisetins, or rye secalins. Rice globulin is more preferred.

Suitable selectable markers for selection in plant cells include, but are not limited to, antibiotic resistance genes, such as kanamycin (nptII), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance. The particular marker gene employed is one which allows for selection of transformed cells as compared to cells lacking the nucleic acid which has been introduced. Preferably, the selectable marker gene is one that facilitates selection at the tissue culture stage, e.g., a nptII, hygromycin or ampicillin resistance gene. Thus, the particular marker employed is not essential to this invention.

In general, a selected nucleic acid sequence is inserted into an appropriate restriction endonuclease site or sites in the vector. Standard methods for cutting, ligating and E. coli transformation, known to those of skill in the art, are used in constructing vectors for use in the present invention.

Plant cells or tissues are transformed with above expression constructs using a variety of standard techniques. It is preferred that the vector sequences be stably integrated into the host genome.

The method used for transformation of host plant cells is not critical to the present invention. For commercialization of the heterologous peptide or polypeptide expressed in accordance with the present invention, the transformation of the plant is preferably permanent, i.e. by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present invention.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

The invention also includes a chimeric gene, comprising:
(i) a promoter that is active in plant cells;
(ii) a first nucleic acid sequence, operably linked to the promoter, encoding a seed storage protein; and
(iii) a second nucleic acid sequence, operably linked to the promoter, encoding a defensin;
wherein the first and second nucleic acid sequences are linked in translation frame and together encode a fusion protein comprising the storage protein and the defensin.

The seed storage protein may be at the N-terminal or C-terminal side of the α-defensin in the fusion protein. It is preferred that the seed storage protein be located at the N-terminal side of the defensin.

The fusion protein may also be engineered to comprise at least one selective purification tag and/or at least one specific protease cleavage site for eventual release of the α-defensin from the seed storage protein fusion partner, fused in translation frame between the α-defensin and the seed storage protein. Preferably, the specific protease cleavage site may comprise enterokinase (ek), Factor Xa, thrombin, V8 protease, Genenase™, α-lytic protease or tobacco etch virus (TEV) protease. The fusion protein may also be cleaved chemically.

### EXAMPLES

### Example 1

### HD5 Gene Synthesis

The codons for genes expressed in rice are rich in guanines (G) or cytosines (C) in the third position of the codons (Huang et al., J. CAASS 1: 73-86 (1990)). Changing low G+C contents to high G+C contents has been found to increase the expression of foreign genes in rice (Huang et al., Molecular Breeding, 10(1-2): 83-94 (2002); Nandi et al., Plant Science 163: 713-22 (2002)). Therefore, two versions of codon-optimized HD5 gene were synthesized based on the codon-usage preference of rice genes.

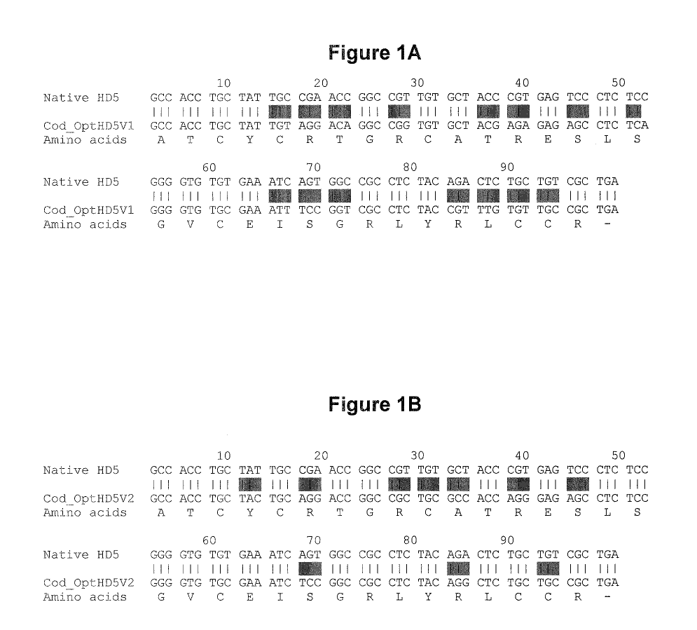

The codons of human α-defensin 5 gene (GenBank accession no. NP_{—}066290) were optimized based on the codon-usage preference of rice genes. In this study, we prepared two versions of codon-optimization for HD5 gene and they were designated as HD5V1 and HD5V2 (FIGS. 1A and 1B), respectively. In HD5V1, the genetic codons for HD5 was selected based mainly on their high frequency of usage in rice genes (http://www.kazusa.or.jp/codonlcgi-bin/showcodon.cgi?species=Oryza+sativa+[gbpln]) after rare codons were eliminated and other potentially unfavorable features associated with codons were taken into consideration (Gustafsson et al., Trends Biotechnol. 22:346-53 (2004)). In HD5V2, the genetic codons for HD5 were optimized in a similar way as for HD5V1 except that the nucleotide in the third position of the codons was selected as guanines (G) or cytosines (C) if applicable.

Each of the two versions of codon-optimized HD5 gene sequences was synthesized de novo and cloned into the pUC57 plasmid with SfoI and XhoI restriction sites engineered upstream and downstream the HD5 gene, respectively, in order to facilitate subsequent cloning. The synthetic HD5 gene sequence was sequence-verified to encode the correct HD5 peptide sequence, and the resulting plasmids harboring two versions of codon-optimized HD5 gene sequences are named pUC-HD5V1 and pUC-HD5V2, respectively.

### Example 2

### Plasmid Construction for Rice Transformation

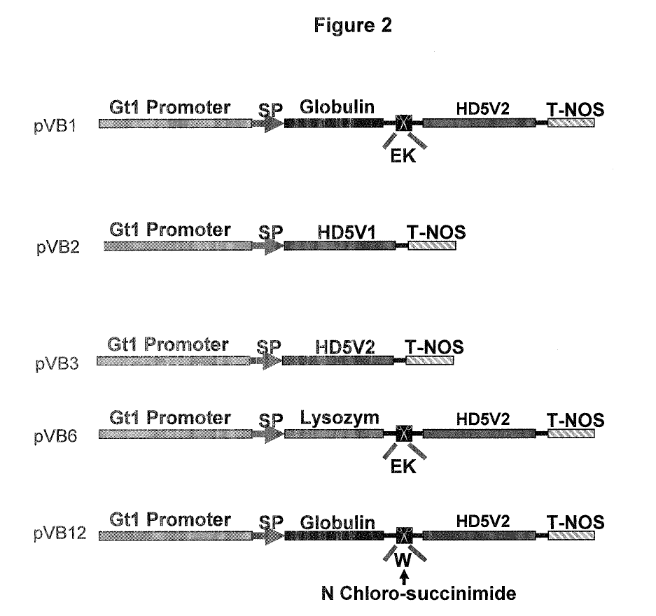

Five plasmids were prepared for expression of HD5 in rice (FIG. 2).

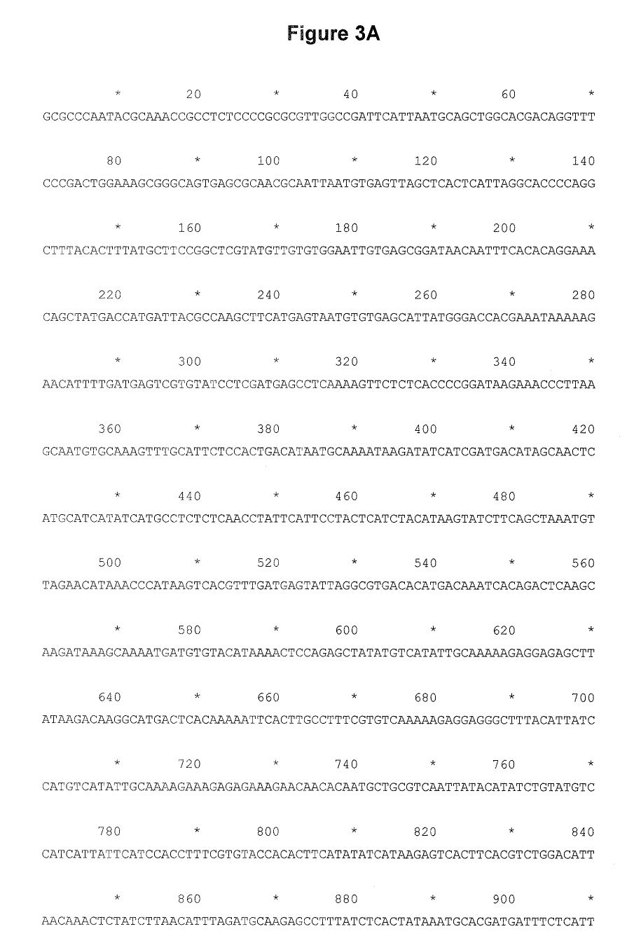

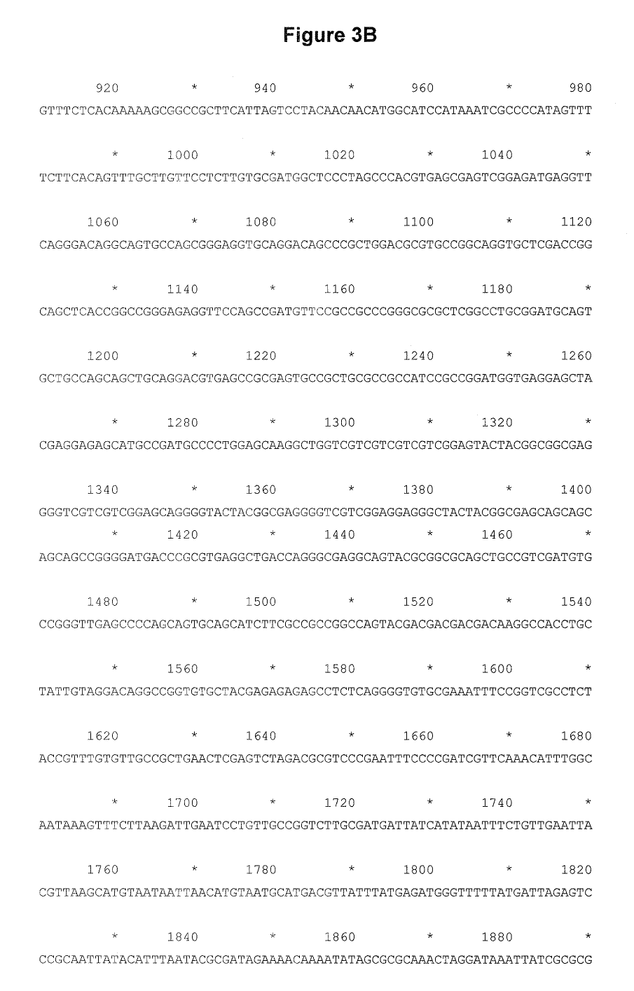

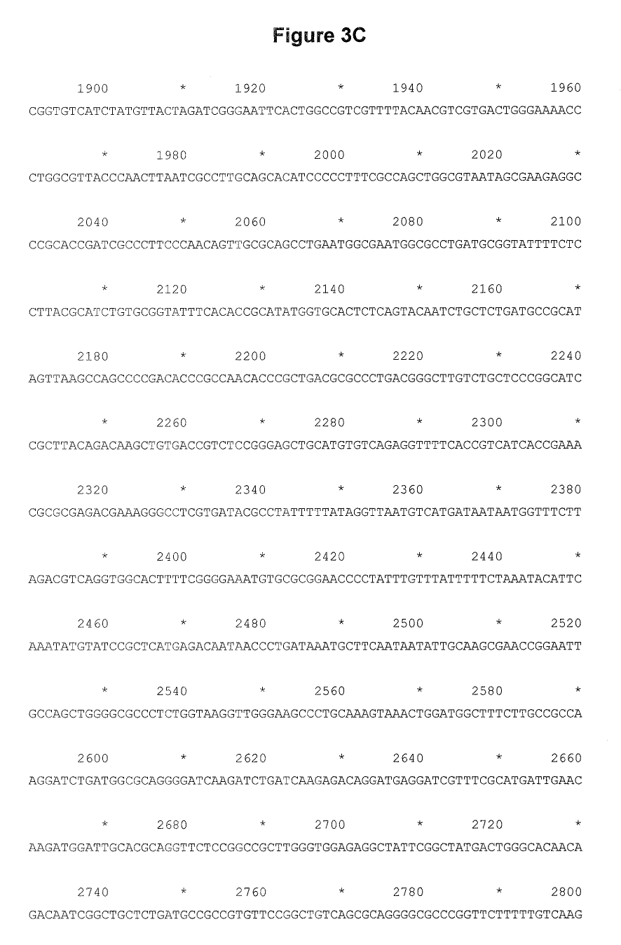

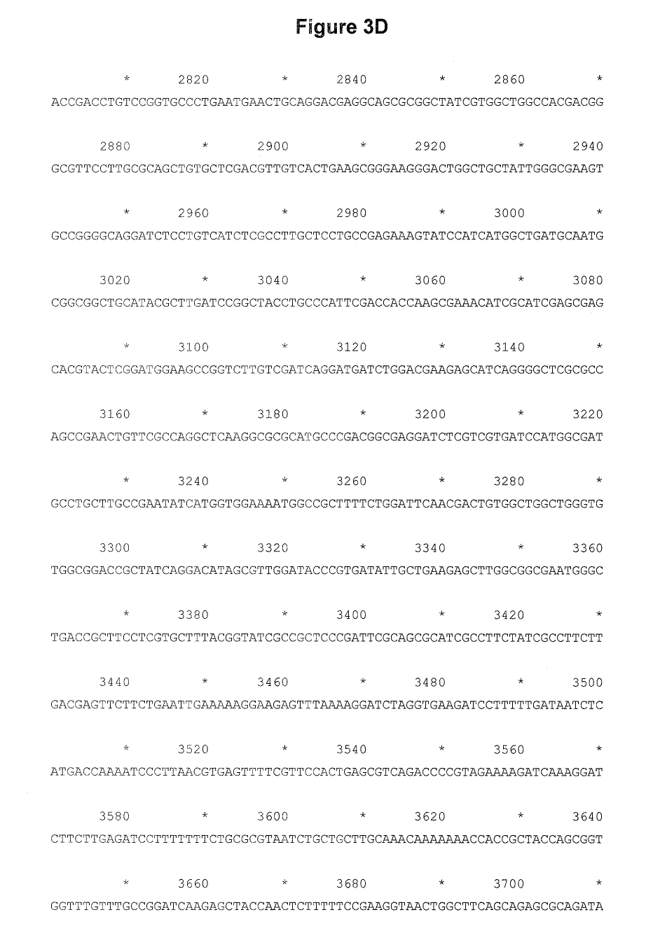

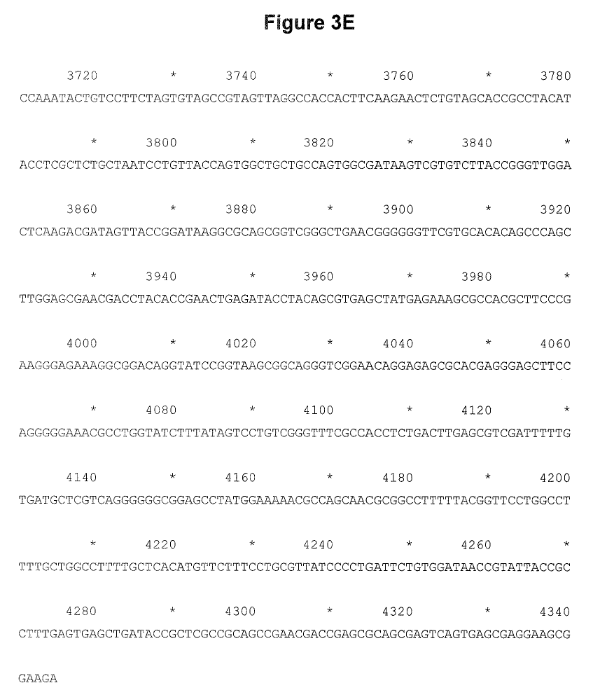

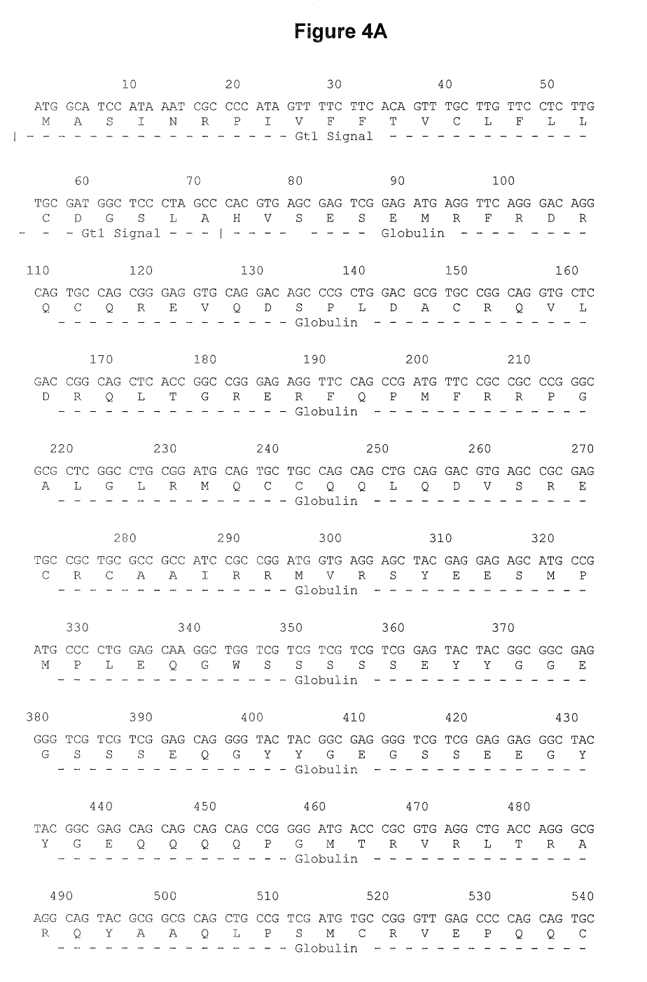

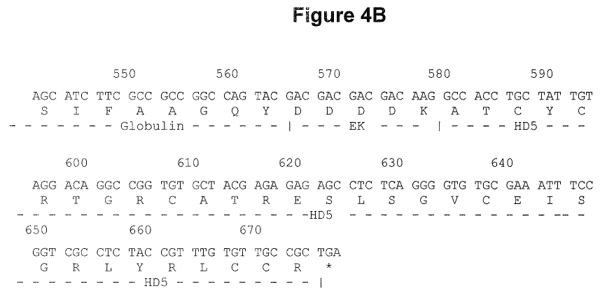

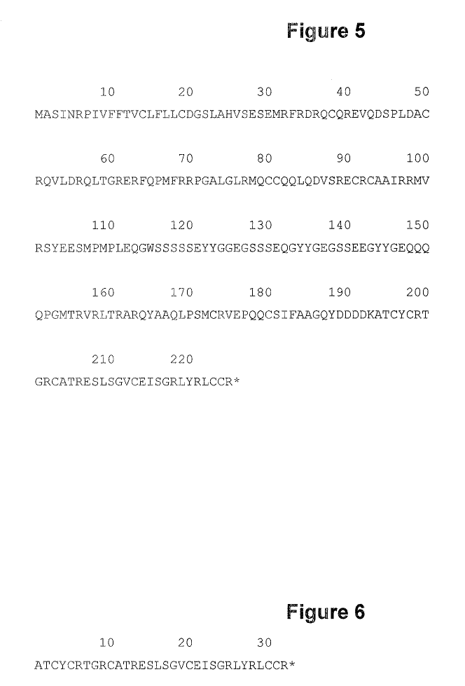

The plasmid pVB1 was constructed using Ventria's plasmid pAP1496 as backbone vector, which contains the Glutelin 1 gene promoter (Gt1) and its signal peptide (Genbank accession no Y00687), rice globulin gene (Genbank accession no. X63990), ITF gene with the addition of enterokinase (EK) cleavage site at the N-terminus, and the NOS terminator (Genbank accession no. AJ007624). The pAP1496 was first opened up by BamHI, which is immediately downstream to the EK site, followed by being blunted with Mung bean nuclease, and then cut by XhoI (upstream the Nos terminator) to remove the ITF gene fragment followed. The removed ITF DNA fragment was then replaced with the HD5V2 gene fragment that was released with XhoI and blunt-cutting SfoI from pUC-HD5V2. Sequence information for plasmid pVB1 are set forth in FIGS. 3 to 6.

The plasmid pVB2 was made as follows: the β-glucuronidase (Gus) gene fragment in Ventria's plasmid pAP1405, which contains the Gt1 promoter and its signal peptide, Gus gene, and the NOS terminator, was removed by double digestion with restriction enzymes NaeI and XhoI, and replaced with the HD5V1 gene sequence released with XhoI and SfoI from pUC-HD5V1.

The plasmid pVB3 were prepared in the same way as for pVB2 except that the HD5 gene nucleotide sequence was version HD5V2 rather than version HD5V1.

For the construction of plasmid pVB6, the Ventria's plasmid pVB5 containing Gt1 promoter, codon-optimized human lysozyme gene (Genbank accession no. J03801), ITF gene, and the NOS terminator served as a starting point. The plasmid pVB5 was first cut by BamHI followed by being blunted with Mung bean nuclease, and then cut by XhoI to remove the ITF DNA fragment and dephosphorylated with calf intestinal alkaline phosphatase (CIAP). Then the XhoI and SfoI human defensin gene fragment from pUC-HD5V2 was introduced for replacing the ITF gene fragment.

The construction of plasmid pVB12 was carried out by using Ventria's plasmid pAPI506 as the base vector. The plasmid pAPI506 contains the Gt1 promoter, rice globulin gene fused with a synthetic 16-amino acid C-terminus lipolytic fragment of human growth hormone (AOD9604) with the addition of a tryptophan residue to the N-terminus as the N Chloro-succinimide cleavage site, and the Nos terminator. To replace the AOD9604 gene fragment with the HD5, the plasmid pAP1506 was mutated to engineer a blunt-cutting Msc I restriction site right after the tryptophan residue through SDM (site-directed mutagenesis) using the below pairs of primers: Forward, 5′ GCCGGCCAGTACTGGCCACTCCGCATCGTGCAG 3′ and Reverse, 5′ CGGCCGGTCATGACCGGTGAGGCGTAGCACGTC 3′. The resulting plasmid (named as pVB8) was cut by XhoI, followed by gel purification, and then partially digested by MscI followed by gel purification to remove the AOD9604 DNA fragment. Then the XhoI and SfoI human defensin gene fragment from pUC-HD5V2 was brought in by ligation to replace AOD9604 fragment.

All the procedures for plasmid subcloning were carried out following the standard molecular cloning procedures (Sambrook et al., Molecular Cloning. A Laboratory Manual. Cold Spring Harbor Laboratory Press (1989)).

### Example 3

### Rice Transformation to Generate Transgenic Plants

Two rice varieties, Taipei 309 and Bengal, were used for gene transformation. The procedure of rice transformation using microprojectile bombardment was performed with the Biolistic PDC-1000/He system (Bio-Rad) as previously described (Huang et al., Plant Science 161: 589-95 (2001)). Briefly, rice seeds were dehusked, sterilized in 20% (v/v) commercial bleach for 20 min and washed with sterile water three times for five min each. The sterilized seeds were placed on rice callus induction (RCI) medium containing N6 salt (Sigma), B5 vitamins (Sigma), 2 mg/L 2,4-D, and 3% sucrose for 10 days to induce callus. Then the primary callus was subcultured on RCI medium for two weeks.

Calli that were 2-4 mm diameter were selected and placed in a 4 cm diameter circle on RCI with 0.3 M mannitol and 0.3 M sorbitol for 24 h before bombardment. The calli were bombarded with 1.5 mg of gold particles (60 ug/ul) coated with 2.5 ug of minimal expression cassette DNAs for expression of hygromycin B (selectable marker gene) and HD5 gene, respectively, at a ratio of 1 to 2 at a helium pressure of 1100 psi. The minimal expression cassette DNAs were linear DNA fragment cut from the plasmid DNA to exclude any vector backbone sequence. After bombardment, the calli were recovered on the same medium plate for 48 h, and then transferred to RCI with 80 mg/L hygromycin B to incubate in the dark at 26° C. for 45 days. Then the hygromycin B-resistant transformants were selected and transferred to RCI (without 2,4-D) with 5 mg/L ABA, 2 mg/L BAP, 1 mg/L NAA for 9 to 12 days in dim light followed by being transferred onto the regeneration medium consisting of RCI without 2,4-D, 3 mg/l BAP, and 0.5 mg/l NAA and cultured under continuous lighting conditions for two to four weeks. After the regenerated plants were 1 to 3 cm high, the plantlets were transferred to rooting medium containing the half concentration of the MS medium (Sigma) plus 1% sucrose and 0.05 mg/l NAA for two weeks to allow the development of roots. Those plants having vigorously growing root systems were transferred to soil in a greenhouse, and were grown to maturity after the insertion of the HD5 gene was confirmed by PCR amplification (see Example 4, below). The transgenic plants that were transferred to the greenhouse are referred to as the “R0 generation” or “R0 plants”.

### Example 4

### Determine Transgenic Plants with HD5 Gene

To confirm the insertion of HD5 expression cassette DNA in rice, PCR analysis of regenerated plants was conducted using the Extract-N-Amp Plant PCR kit (Sigma) with pair of primers of which one was specific to the HD5 gene and the other was specific to the rice globulin gene. PCR primer design and PCR conditions followed general molecular biology procedures in order to reduce primer-dimer formation and increase amplification efficiency. Non-transgenic plants were used as controls. More than 100 transgenic plants (R0) were identified from each gene construct and were transferred to a closed greenhouse at different stages (Table 1).

TABLE 1
Transgenic rice plants for each plasmid construct.

| Plasmid | Host | Number of Transgenic Plants |
|---|---|---|
| pVB1 | TP309 | 154 |
| | Bengal | 237 |
| PVB2 | TP309 | 162 |
| | Bengal | 33 |
| PVB3 | TP309 | 146 |
| | Bengal | 106 |

### Example 5

### Plant Culture in Greenhouse

Once the transgenic rice plants were transferred to the greenhouse, they were transplanted into 8-inch pots. Fertilizer was added and rice growth was monitored. Water and pest management were carried out according to good agricultural practices, in order to ensure healthy growth of the transgenic rice plants. The greenhouse was maintained at temperatures of 30° C. (daytime) and 25° C. (nighttime). Depending on the date and season, supplemental light was added to provide additional light in the daytime. The seeds set by these transgenic plants are called “R1 seeds.” Upon harvest, rice R1 seeds were dried in an incubator set at 5000 for 5 days, during which time the moisture content was reduced from about 20% to less than 14%.

### Example 6

### Recombinant HD5 Expression Analysis of Transgenic R1 Seeds

To screen the expression of HD5 in rice grains, 10 randomly-picked transgenic R1 seeds from each transgenic R0 plant were pooled and assessed using sodium dodecylsulfate-polyacrylamide gel electrophoresis (SDS-PAGE). The 10 pooled dehusked-seeds were ground into fine powder using mortar and pestle, mixed with 5 ml of SDS extraction buffer (65 mM Tris-HCl, pH 6.8, 2% SDS, and 2% 2-mercaptoethonal), and incubated at room temperature for 1 h followed by centrifugation. The supernatant protein extract was separated on the precasted Tris-Glycine SDS-PAGE gels (Invitrogen) followed by Coomassie Blue staining, and the HD5-expressing transgenic lines were identified.

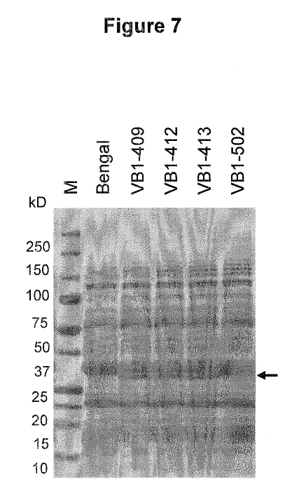

As seen in FIG. 7, a single protein band corresponding to the fusion protein of rice globulin and human defensin was identified in some transgenic lines while this band was absent in the non-transgenic control. This band was determined to correspond to HD5 fusion protein via Western blot. Nearly half of the transgenic lines from cultivars Taipei 309 (46%) and Bengal (47%) were shown to be positive for the HD5 fusion protein. This number is possibly underestimated as some plants with lower expression levels might not be able to be detected through the SDS-PAGE analysis.

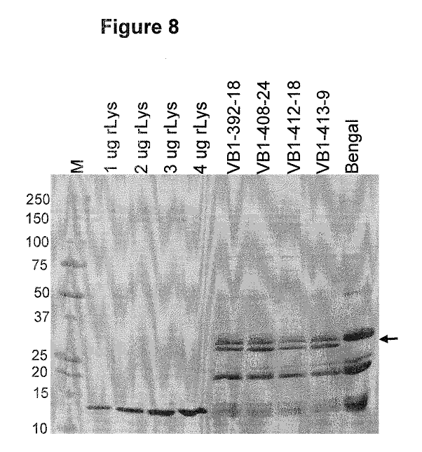

To estimate the expression level of HD5 in different transgenic lines, the seed with the highest expression of human defensin from each line based on the intensity of the HD5 fusion band was chosen as the representative for quantification. The protein extracts of the selected seeds were resolved on the SDS-PAGE in parallel with the titration of the known amount of recombinant human lysozyme (rhLZ) (see FIG. 8). Then the amount of target protein was estimated by comparing the intensity of the target protein band with that of rhLZ using the Kodak 1D image analysis program.

Expression levels varied from line to line. The expression level of defensin fusion in one of the best lines, VB1-408-24, was estimated to be 9 mg/g⁻¹ or 0.9% of dehusked rice grain weight. As the molecular weight of defensin (3.5 KD) is one sixth the molecular weight of the fusion protein (23 KD), the calculated expression level of defensin in rice grain was approximately 0.1% of rice grain weight.

### Example 7

### Verification of HD5 Gene Expression in Transgenic Rice R1 Seeds

Western blot analysis was used to verify the extra band in transgenic rice but not present in non-transgenic control is truly HD5 fusion protein. The protein extracts were resolved on 4-20% Tris-glycine SDS-PAGE gels. The gels were equilibrated in transfer buffer (25 mM Tris pH 8.3, 192 mM glycine, 20% (v/v) methanol) for 5 minutes and then transferred to nitrocellulose membrane using a Mini Trans-Blot Cell (Bio-Rad). The membrane was blocked overnight in blocking buffer (3% BSA, 0.02% NaN₃) at 4° C. The membrane was incubated in primary antibody (anti-hDefensin 5-α, US Biological, or anti-rice globulin, prepared in house) at a 1:2000 concentration in blocking buffer for 2 h at room temperature. The membrane was washed four times for 5 minutes with TBST (25 mM Tris pH 7.4, 135 mM NaCl, 0.05% Tween) and then incubated with secondary antibody (anti-rabbit HRP, Rockland) at a concentration of 1:15000 in TBST for 45 minutes at room temperature followed by washing with TBST four times for 5 minutes. The membrane was then incubated with ECL. reagent (Pierce) for 1 minute followed by exposure to CL-Xposure (Pierce) autoradiography film.

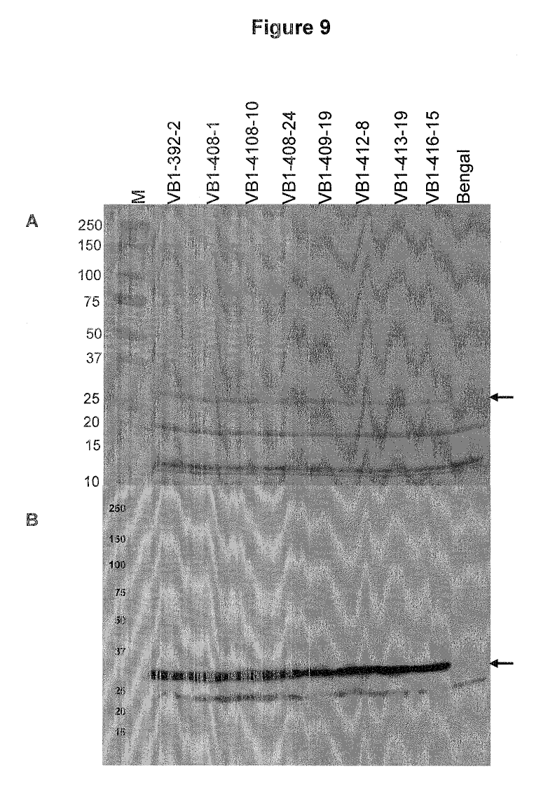

When the Western blot was probed with anti-defensin antibody, four bands were detected in the transgenic lines, but only the top band was absent in the non-transgenic line and shown the same molecular size as that in the Coomassie Blue-stained SDS-PAGE gel (FIG. 9A). As the three lower bands were also seen in non-transgenic lines, it is unlikely that these bands were from the proteolytic cleavage of the fusion protein, instead they may represent the cross-reactivity of anti-defensin antibody with rice proteins. In contrast to the multiple bands from the antidefensin antibody, the anti-globulin antibody detected two expected bands in the transgenic lines as opposed one band in the non-transgenic line (FIG. 9B). The top band in the transgenic lines, which was not only absent in the non-transgenic line but with the same molecular size of the band in SDS-PAGE gel, was the fusion protein of HD5 and rice globulin. The lower band in the transgenic lines, which was also seen in the non-transgenic line, corresponded to the endogenous globulin protein.

### Example 8

### Segregation of HD5 Gene in Transgenic Rice R1 Seeds

The segregation analysis of R1 seeds of the positive transgenic lines expressing the HD5 was carried out by performing expression analysis of individual seeds. 24 to 40 R1 seeds were randomly selected for each HD5-expressing transgenic line. The single seed was cut in half cross-sectionally so the embryo-containing half was saved to proceed to next generation while the endosperm-half without embryo was used for protein extraction. Each embryo-less half seed was separately put in 500 ul of SDS extraction buffer in a well of 96 deep-well plate and soaked for 3 h at room temperature followed by adding two 10 mm diameter steel beads per well and vortexing with Geno/Grinder 2000 (SPEX CertiPrep, Metuchen, N.J.) for 20 min at 1300 strokes/min. Then the mixture was centrifuged at 4,000 rpm for 20 min at 4 C in a microplate-centrifuge (Eppendorf), and the crude protein extract was transferred to a new microplate for SDS-PAGE analysis. The number of positive and negative seeds in terms of expressing HD5 was investigated for each line, and Chi-square statistical test was performed to examine the inheritance model of transgene.

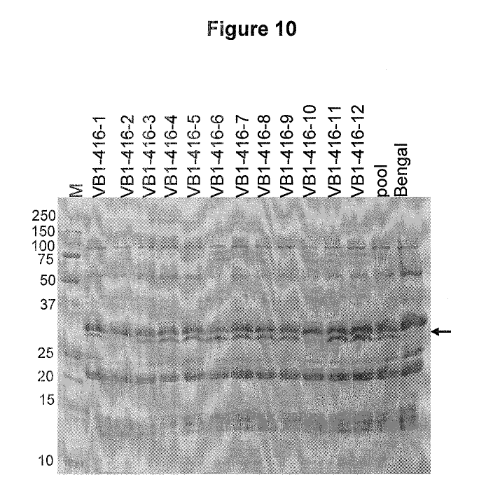

The SDS-PAGE analysis of the embryo-less half-seeds showed variation of expression levels of HD5 among individual seeds from the same transgenic line (FIG. 10). For all the lines analyzed, the observed segregation ratio of positive to negative fitted a 3:1 segregation ratio (P≦0.05). The monogenic inheritance of transgene provided advantages in terms of selecting stable homozygous lines at a fast pace. The saved embryo-containing half-seeds corresponding to the embryoless half-seeds that showed high expression of HD5 were sterilized and germinated on half-strength MS medium, and were then planted in soil for advancement to the next generation.

TABLE 2
Segregation analysis of defensin-expressing transgenic lines.

| | No. of R1 | | | |
|---|---|---|---|---|
| Transgenic | seeds | No. of positive | No. of negative | χ2 critical |
| line ID | analyzed | R1 seeds | R1 seeds | value |
| VB1-392 | 40 | 33 | 7 | 1.2 |
| VB1-407 | 32 | 21 | 11 | 1.5 |
| VB1-408 | 40 | 33 | 7 | 1.2 |
| VB1-409 | 40 | 27 | 13 | 1.2 |
| VB1-412 | 24 | 20 | 4 | 0.89 |
| VB1-413 | 24 | 19 | 5 | 0.22 |
| VB1-416 | 40 | 26 | 14 | 2.13 |

### Example 9

### Antimicrobial Activity Analysis of rHD5

The antimicrobial activity of recombinant α-defensin 5 (rHD5) preparation will be analyzed using two approaches. First, rHD5 will be tested using a flow cytometry procedure. Recombinant HD5 as prepared above and protein extract from non-transgenic rice flour will be normalized to protein concentration as determined by Bradford assay. The protein preparations will be added to mid-log growth phase suspension of E. coli (ATCC 25922) and the mixture will be incubated at 37° C. in a final volume of 100 μl 1:6 diluted Schaedler Broth (BD, Sparks, Calif.). Bacterial suspensions incubated with vehicle (0.01% acetic acid) and protein from non-transgenic flour will serve as negative controls. Standard antibiotics will be used as the positive control. After 120 minutes, bis-(1,3-dibutylbarbituric acid) trimethine oronol (DiBAC4(3), Molecular Probes, Eugene, Oreg.), a dye sensitive to membrane potential, will be added at a concentration of 1 μg/ml. Bacterial pellets will be isolated using centrifugation, resuspended in 300 μl FACSFlow™ (BD), and analyzed by flow cytometry using a FACSCalibur™ (BD). Antimicrobial activity is determined as a percentage of depolarized bacteria compared to untreated non-transgenic controls.

Rapid growing mid-log phase E. coli will be harvested and diluted to 5×105/ml. Recombinant HD5 and soluble protein from non-transgenic plants will be added to bacterial culture at a final concentration from 500 ng/ml to 500 pg/ml. The mixture will be incubated at 37° C. for 120 to 240 minutes. After incubation, the culture mixture will be diluted and plated on LB plates and incubated overnight at 37° C. Colony forming units (CFU) will then be determined. Antimicrobial activity of rHD5 will be determined by comparing the reduction of CFU from rHD5 plates to CFU from non-transgenic control.

The result will be a transgenic rice line expressing a high level of rHD5, which will provide the base for the development of compositions for the management of intestinal diseases.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

What is claimed:

1. A transgenic monocot seed that yields, by extracting ground seed with an aqueous medium, a total soluble protein fraction containing at least 0.1% by total soluble protein weight of a defensin.

2. The seed of claim 1, wherein the defensin is selected from the group consisting of human α-defensin 1, human α-defensin 2, human α-defensin 3, human α-defensin 4, human α-defensin 5, and human α-defensin 6.

3. The seed of claim 1, whose total soluble protein fraction contains at least 0.3% by total soluble protein weight of a human defensin.

4. The seed of claim 1 which is a mature, transgenic rice, corn, barley or wheat seed.

5. The seed of claim 4 which is a mature, transgenic rice seed.

6. A method of producing a defensin in plant seeds, comprising the steps of:
(a) transforming a plant cell with a chimeric gene comprising
(i) a maturation-specific protein promoter from a plant,
(ii) a first nucleic acid sequence, operably linked to the promoter, encoding a seed-specific signal sequence capable of targeting a polypeptide linked thereto to seed endosperm, and
(iii) a second nucleic acid sequence, linked in translation frame with the first nucleic acid sequence, encoding a defensin, wherein the first nucleic acid sequence and the second nucleic acid sequence together encode a fusion protein comprising an N-terminal signal sequence and the defensin;
(b) growing a plant from the transformed plant cell for a time sufficient to produce seeds containing the defensin; and
(c) harvesting the seeds from the plant.

7. The method of claim 6, wherein the plant cell is from a monocot plant.

8. The method of claim 7, wherein the plant is a cereal selected from the group consisting of rice, barley, wheat, oat, rye, corn, millet, triticale and sorghum.

9. The method of claim 8, wherein the plant is rice.

10. The method of claim 6, wherein the defensin comprises about 0.1% or greater of the total soluble protein in the seeds.

11. The method of claim 6, wherein the defensin comprises about 0.3% or greater of the total soluble protein in the seeds.

12. The method of claim 6, wherein the defensin is selected from the group consisting of human α-defensin 1, human α-defensin 2, human α-defensin 3, human α-defensin 4, human α-defensin 5, and human α-defensin 6.

13. A method of producing at least one human a-defensin, comprising the steps of:
a) providing a plant cell transformed with a vector containing a promoter and a gene, operably linked to the promoter, encoding a human α-defensin,
b) growing a plant from the transformed plant cell for a time sufficient to produce seeds,
c) harvesting the mature seeds, and optionally
d) purifying the desired human α-defensin from the seeds or seed product.

14. The method of claim 13, wherein the plant cell is from a monocot plant.

15. The method of claim 14, wherein the plant is a cereal selected from the group consisting of rice, barley, wheat, oat, rye, corn, millet, triticale and sorghum.

16. The method of claim 15, wherein the plant is rice.

17. A chimeric gene, comprising:
(i) a promoter that is active in plant cells;
(ii) a first nucleic acid sequence, operably linked to the promoter, encoding a seed storage protein; and
(iii) a second nucleic acid sequence, operably linked to the promoter, encoding a human α-defensin:
wherein the first and second nucleic acid sequences are linked in translation frame and together encode a fusion protein comprising the storage protein and the human a-defensin.

18. A vector, comprising (i) a promoter from a monocot plant gene that has upregulated activity during seed maturation, (ii) a first DNA sequence, operably linked to said promoter, encoding a monocot plant seed-specific signal sequence capable of targeting a polypeptide inked thereto to monocot plant seed endosperm, and (iii) a second DNA sequence, linked in translation frame with the first DNA sequence, encoding a human defensin, wherein the first DNA sequence and the second DNA sequence together encode a fusion protein comprising an N-terminal signal sequence and the human defensin.

19. A method of producing seeds that express a defensin and a seed storage protein as a fusion partner, the method comprising:
(a) transforming a plant cell with a chimeric gene comprising:
(i) a promoter that is active in plant cells;
(ii) a first nucleic acid sequence, operably linked to the promoter, encoding a seed storage protein;
(iii) a second nucleic acid sequence, operably linked to the promoter, encoding a defensin; and
(iv) optionally a signal sequence, wherein the first and second nucleic acid sequences and the optional signal sequence are linked in translation frame and together encode a fusion protein comprising the storage protein, the defensin and the optional signal sequence;
(b) growing a plant from the transformed plant cell for a time sufficient to produce seeds containing the fusion protein; and
(c) harvesting the seeds from the plant.

20. The method of claim 19, wherein the defensin is selected from the group consisting of human α-defensin 1, human α-defensin 2, human α-defensin 3, human α-defensin 4, human α-defensin 5, and human α-defensin 6.

21. A method of treating an intestinal disease in a patient suffering therefrom, comprising the step of orally administering to said patient an oral formulation comprising a composition made from the seed of claim 1.

22. A method of topically administering one or more defensins to a subject, comprising the step of topically administering to said subject a formulation comprising a composition made from the seed of claim 1.

23. The method of claim 22, wherein the one or more defensins are administered for skin care.

24. The method of claim 22, wherein the one or more defensins are administered for wound care.
